(19) United States
(12) Patent Application Publication
(10) Pub. No.: US 2026/0215862 A1
(43) Pub. Date: Jul. 30, 2026

(54) SURGICAL ROBOTIC SYSTEM AND METHOD FOR CALIBRATION OF JAWED INSTRUMENTS

(71) Applicant: Covidien LP, Mansfield, MA (US)

(72) Inventors: David J. Boteler, Boulder, CO (US); Haralambos P. Apostolopoulos, Westminster, CO (US); Jessica Brianne Arehart, Boulder, CO (US); Christopher L. Tschudy, Arvada, CO (US); Connor D. Roberts, Arvada, CO (US); James J. Butch, Denver, CO (US); Dylan R. Kingsley, Broomfield, CO (US); Matthew D. Straka, Arvada, CO (US)

(21) Appl. No.: 19/146,291

(22) PCT Filed: Jan. 22, 2024
(86) PCT No.: PCT/IB2024/050589

§ 371 Date: Jul. 8, 2025

Related U.S. Application Data
(60) Provisional application No. 63/440,950, filed on Jan. 25, 2023.
(60) Provisional application No. 63/461,964, filed on Apr. 26, 2023.
(60) Provisional application No. 63/620,357, filed on Jan. 12, 2024.

(51) Int. Cl. A61B 34/30; A61B 17/00; A61B 17/29; A61B 90/00

(52) U.S. Cl. CPC A61B 34/30; A61B 17/29; A61B 90/06; A61B 2017/00725; A61B 2017/2944; A61B 2034/301; A61B 2090/066

(57) ABSTRACT

A surgical robotic system includes a robotic arm and an instrument for use in surgical procedures. The instrument includes a coupler, a drive rod, a spring, and a pair of opposing jaws. The robotic arm includes an instrument drive unit having a motor, a torque sensor, and a position sensor. The controller of the system is configured to actuate the motor to move the jaws to a closed position and to receive torque and position data from the sensors. The controller then determines a change in slope point from the data and identifies a calibrated coupler position based on the change in slope point. The system is able to control movement of the jaws based on the calibrated coupler position, providing precise and accurate surgical procedures.

### CROSS-REFERENCE TO RELATED APPLICATIONS

This application claims the benefit of U.S. Provisional Patent Application Ser. No. 63/440,950, filed Jan. 25, 2023, U.S. Provisional Patent Application Ser. No. 63/461,964, filed Apr. 26, 2023, and U.S. Provisional Patent Application Ser. No. 63/620,357, filed Jan. 12, 2024 the entire content of which are incorporated herein by reference.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. Surgical robotic systems are used with a variety of jawed surgical instruments, such as graspers, cutters, electrosurgical vessel sealers, etc.

### SUMMARY

According to one embodiment of the present disclosure, a method for calibrating a surgical robotic instrument is disclosed. The method includes actuating a motor to rotate a coupler of a surgical robotic instrument, where rotation of the coupler moves a drive rod disposed within the instrument in a longitudinal direction to compress a spring and approximate at least one jaw of a pair of opposing jaws of the instrument to a closed position. The method also includes measuring torque imparted by the motor and rotational position of the coupler during actuation of the motor to approximate the at least one jaw of the pair of opposing jaws of the instrument to the closed position. The method further includes determining a change in slope point from a plot of the torque and the rotational position. The method additionally includes identifying a coupler position corresponding to the change in slope point as a calibrated coupler position and controlling movement of the at least one jaw based on the calibrated coupler position.

Implementations of the above embodiment may include one or more of the following features. According to one aspect of the above embodiment, the change in slope point may be indicative of a start of compression of the spring. The method may further include loading one or more calibration parameters for controlling movement of the at least one jaw based on the calibrated coupler position. The method may also include setting a closed position setpoint based on the calibrated coupler position. The calibration parameter may be a closure distance value. Setting the closed position setpoint may include adding the closure distance value to the calibrated coupler position. The method may also include setting an opened position setpoint based on the calibrated coupler position. The calibration parameter may be an opening distance value. Setting the opened position setpoint may include adding the opening distance value to the calibrated coupler position.

According to another embodiment of the present disclosure, a surgical robotic system is disclosed. The system includes a robotic arm having an instrument drive unit having a motor, a torque sensor, and a position sensor. The surgical robotic system also includes an instrument having a coupler configured to engage the motor, a drive rod longitudinally movable by the coupler, a spring compressed by the coupler during movement of the drive rod, and a pair of opposing jaws movable by the drive rod to a closed position. The system further includes a controller configured to actuate the motor to approximate the at least one jaw of the pair of opposing jaws to the closed position, receive torque imparted by the motor from the torque sensor and rotational position of the coupler from the position sensor, determine a change in slope point from a plot of the torque and the rotational position, identify a coupler position corresponding to the change in slope point as a calibrated coupler position, and control movement of the at least one jaw based on the calibrated coupler position.

Implementations of the above embodiment may include one or more of the following features. According to one aspect of the above embodiment, the change in slope point may be indicative of a start of compression of the spring. The instrument may further include a storage device storing one or more calibration parameters. The controller may set a closed position setpoint based on the calibrated coupler position. The calibration parameter may be a closure distance value. Setting the closed position setpoint may include adding the closure distance value to the calibrated coupler position. The controller may also set an opened position setpoint based on the calibrated coupler position. The calibration parameter may be an opening distance value. Setting the opened position setpoint may include adding the opening distance value to the calibrated coupler position.

According to a further embodiment of the present disclosure, a method for calibrating a surgical robotic instrument is disclosed. The method includes actuating a motor to rotate a coupler of a surgical robotic instrument, where rotation of the coupler moves a drive rod disposed within the instrument in a longitudinal direction to approximate at least one jaw of a pair of opposing jaws of the instrument until the pair of opposing jaws contact each other. The method also includes measuring torque imparted by the motor during actuation of the motor approximating the at least one jaw of the pair of opposing jaws of the instrument to the closed position. The method further includes determining the pair of opposing jaws contact each other based on measured torque. The method also includes actuating the motor to rotate the coupler of the surgical robotic instrument, where rotation of the coupler moves the drive rod to compress a spring and approximate the at least one jaw of the pair of opposing jaws of the instrument to a closed position. The method further includes measuring torque imparted by the motor and rotational position of the coupler during actuation of the motor approximating the at least one jaw of the pair of opposing jaws of the instrument to the closed position and compressing the spring. The method additionally includes determining a change in slope point from a plot of the torque and the rotational position and identifying a coupler position corresponding to the change in slope point as a calibrated coupler position and controlling movement of the at least one jaw based on the calibrated coupler position.

Implementations of the above embodiment may include one or more of the following features. According to one aspect of the above embodiment, the method may further include loading a closure distance value and an opening distance value, setting a closed position setpoint by adding the closure value to the calibrated coupler position, setting an opened position setpoint by adding the opening value to the calibrated coupler position, and controlling movement of the pair of opposing jaw members between the closed position setpoint and the opened position setpoint.

### BRIEF DESCRIPTION OF THE DRAWINGS

Various embodiments of the present disclosure are described herein with reference to the drawings wherein:

FIG. 1 is a perspective view of a surgical robotic system including a control tower, a console, and one or more surgical robotic arms, each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a perspective view of a surgical instrument provided in accordance with the present disclosure configured for mounting on a robotic arm of a robotic surgical system;

FIG. 8 is a front, perspective view of a proximal portion of the surgical instrument of FIG. 7 with an outer shell removed;

FIG. 9 is a rear, perspective view of the proximal portion of the surgical instrument of FIG. 7 with the outer shell removed;

FIG. 10 is a front, perspective view of the proximal portion of the surgical instrument of FIG. 7 with the outer shell and additional internal components removed;

FIG. 11 is a flow chart of a method of verifying operation of a surgical robotic instrument according to an embodiment of the present disclosure;

FIG. 12 is a flow chart of a method for calibrating the surgical robotic instrument according to an embodiment of the present disclosure;

FIG. 13 is a plot of torque and rotational displacement of a jaw coupler during the calibration method of FIG. 12 according to an embodiment of the present disclosure;

FIG. 14 is an enlarged portion of the plot of FIG. 13;

FIG. 15 is a perspective view of a surgical instrument according to another embodiment of the present disclosure;

FIGS. 16A and B are perspective views of an end effector of the surgical instrument of FIG. 15 in open and closed configurations;

FIG. 17 is a perspective view of a handle controller according to one embodiment of the present disclosure;

FIG. 18 is a plot of paddle feedback force as a function of paddle opening angle for latch operation according to one embodiment of the present disclosure;

FIG. 19 is a flow chart of a method for providing force feedback based on the plot of FIG. 18 according to one embodiment of the present disclosure;

FIG. 20 is a plot of paddle feedback force as a function of paddle opening angle based on spring compression position according to another embodiment of the present disclosure;

FIG. 21 is a flow chart of a method for providing force feedback based on the plot of FIG. 20 according to one embodiment of the present disclosure;

FIG. 22 is a plot of paddle feedback force as a function of paddle opening angle based on spring compression position and latch operation according to a further embodiment of the present disclosure; and

FIG. 23 is a flow chart of a method for calibrating the surgical robotic instrument according to another embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives operator input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands to control one or more actuators of the robotic arm, which would, in turn, move the robotic arm and the instrument in response to the movement commands.

The instrument is a forceps having a pair of opposing jaws with one or both of the jaws being movable relative to each other. In embodiments, the forceps may be electrosurgical forceps configured to seal tissue, e.g., blood vessel(s). The jaws are actuated by a drive rod that is engaged by a spring to provide for a consistent pressure applied by the opposing jaws on the tissue.

Upon coupling the instrument to the robotic arm, the robotic system accesses instrument data, e.g., from a storage device of the instrument. The instrument data includes various parameters pertaining to the instrument. The data includes motor displacement (e.g., rotational displacement) for overcoming the preloading of the spring during jaw closure. The data may be obtained during manufacturing and testing of the instrument by recording the torque from one or more torque sensors and rotational displacement of motor(s) actuating the jaws. The torque and displacement data may be used to generate a torque/displacement curve of the jaw closure action and to identify the number of rotations of the motor for overcoming the preloading of the spring and to enter a linear compression region of the curve.

The robotic system also calibrates the surgical instrument. Calibration may include moving the end effector of the instrument about each degree of freedom (e.g., pitch, yaw, jaw angle, rotation of the entire instrument, etc.) until a mechanical limit or another setpoint is reached. The end effector is also calibrated by closing the jaws. During this process, the spring of the device is compressed to apply a force between the jaws. The robotic system measures and records the torque from one or more torque sensors and rotational displacement of motor(s) actuating the jaws. The torque and displacement data may be used to generate a torque/displacement curve of the jaw closure action and to identify the number of rotations of the motor for overcoming the preloading of the spring and to enter a linear compression region of the curve.

The robotic system then compares the motor displacement value from the storage device to the motor displacement value obtained during calibration to determine if there is a difference. A sufficiently large difference, i.e., above a preset threshold, may be indicative of mechanical failure of one or more components of the instrument, e.g., damaged drive rod or spring. In response to the difference, the robotic system may prevent any use of the instrument as well as write to the storage device to prevent the use of the instrument by any other robotic system.

This verification feature may also be used detect damaged devices or devices not in an identical factory state. In addition, this feature would also allow for verifying functionality of the instrument, e.g., if damage is believed to have occurred. Thus, the verification may occur automatically or in response to an operator command during use of the instrument by the robotic system to determine whether the instrument should continue being used in the procedure. The verification feature may further be used to identify operator errors during calibration, e.g., the presence of extraneous materials being placed between or around the jaws.

The robotic system is also configured to identify the displacement at which the spring begins to be compressed based on an inflection of the curve (e.g., also known as “knee” location due to its similarity to a bent knee joint). The spring compression position is also directly related to the jaw angle (i.e., an aperture of the jaws). The jaw angle may then be used by the robotic system to determine the size of the object being grasped by the jaws, e.g., vessel size. This information may be implemented in other control algorithms of the robotic system, e.g., electrosurgical energy delivery algorithms during vessel sealing, grasping, suturing, etc. Furthermore, the jaw angle may also be used to determine the size of other objects, such as recognizing risk of blade trap or clamping on other objects, e.g., staples.

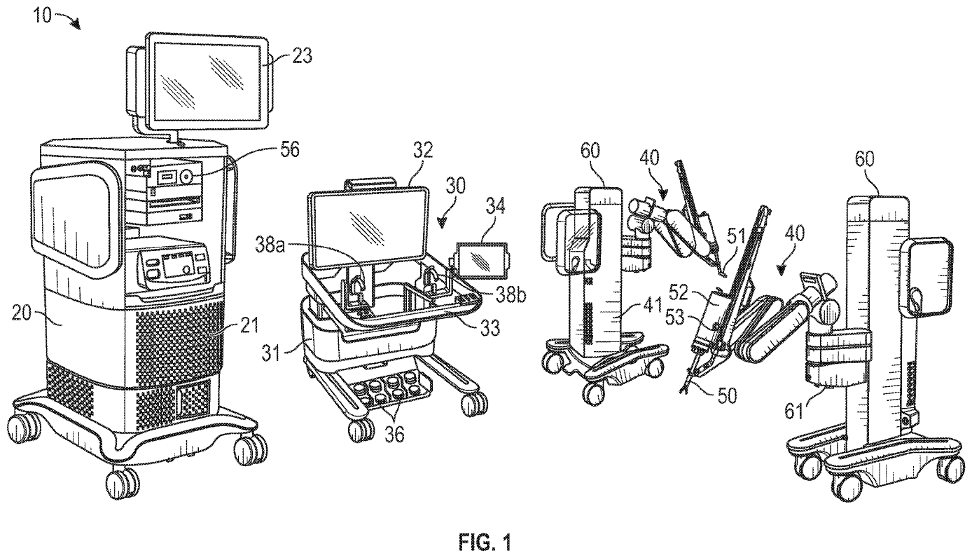

With reference to FIG. 1, a surgical robotic system 10 includes a control tower 20, which is communicatively coupled to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by a camera 51 disposed on the robotic arm 40, and a second display 34, which displays a user interface for controlling the surgical robotic system 10. The first display 32 and the second display 34 may be touchscreens allowing for displaying various graphical user inputs selectable or movable by the user.

The surgeon console 30 also includes a plurality of user interface devices, such as foot pedals 36 and a pair of handle controllers 38a and 38b, which are used by a user to remotely control the robotic arms 40. The surgeon console further includes an armrest 33 used to support clinician's arms while the clinician is operating the handle controllers 38a and 38b.

The control tower 20 can also include a display 23, which may be a touchscreen, and outputs on the graphical user interfaces (GUIs). The control tower 20 also acts as an interface between the surgeon console 30 and one or more of the robotic arms 40. In particular, the control tower 20 is configured to control the robotic arms 40, such as to move the robotic arms 40 and the corresponding surgical instrument 50, based on a set of programmable instructions and/or input commands from the surgeon console 30. In response to the instructions and/or input, the robotic arms 40 and the surgical instrument 50 execute a desired movement sequence in response to input from the foot pedals 36 and the handle controllers 38a and 38b. The system 10 can be configured so that the foot pedals 36 may be used to affect one or more of a wide variety of system functions, such as to enable and lock the hand controllers 38a and 38b, reposition camera movement, and activate/deactivate an electrosurgical instrument. In particular, the foot pedals 36 may be used to perform a clutching action on the hand controllers 38a and 38b. Clutching is initiated by pressing one of the foot pedals 36, which disconnects (i.e., prevents movement inputs from) the hand controllers 38a and/or 38b such that the robotic arm 40 and corresponding instrument 50 or camera 51 are not actuated. This allows the user to reposition the hand controllers 38a and 38b without moving the robotic arm(s) 40 and the instrument 50 and/or camera 51. This is useful when reaching control boundaries of the surgical space, for instance.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency (RF), optical, Wi-Fi, Bluetooth (an open wireless protocol for exchanging data over short distances, using short-length radio waves, from fixed and mobile devices, creating personal area networks (PANs), ZigBee® (a specification for a suite of high-level communication protocols using small, low-power digital radios based on the IEEE 122.15.4-1203 standard for wireless personal area networks (WPANs)).

The computers 21, 31, 41 may include any suitable processor (not shown) connected operably to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure, such as a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

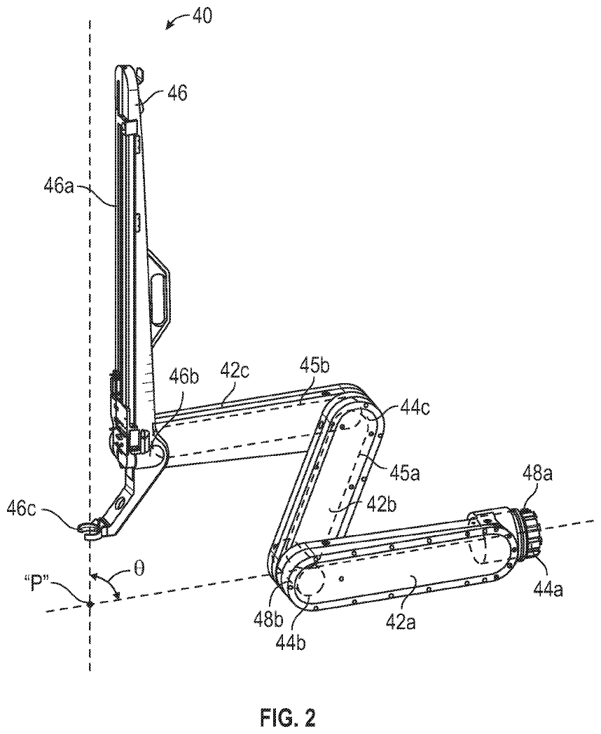

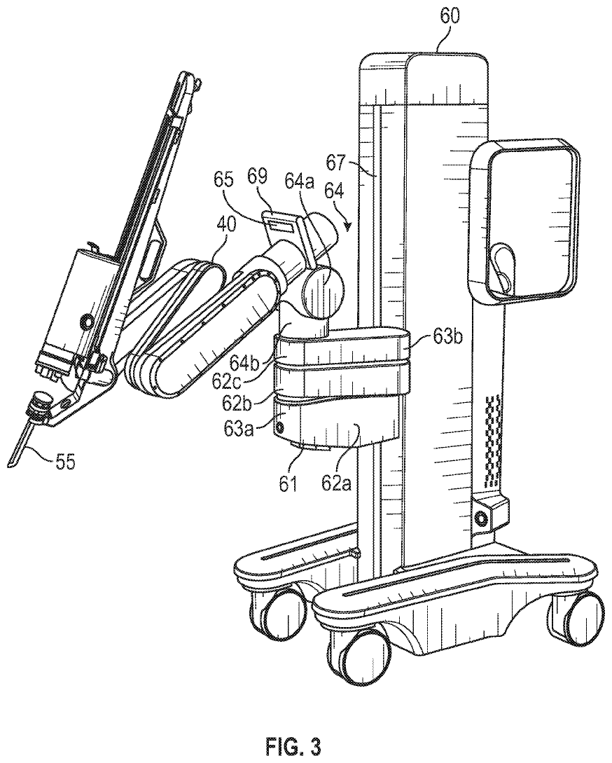

With reference to FIG. 2, each of the robotic arms 40 may include a plurality of links 42a, 42b, 42c, which are interconnected at joints 44b and 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61 and, thereby, of the robotic arms 40 mounted on the setup arm 61. The movable cart 60 also includes a display 69 for displaying information pertaining to the robotic arm 40. In embodiments, the robotic arms 40 may include any type and/or number of joints.

With further reference to FIG. 3, the setup arm 61 includes a first link 62a, a second link 62b, and a third link 62c, which provide for lateral maneuverability of the robotic arms 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in corresponding lateral planes, which are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis, which is perpendicular to a plane defined by the third link 62c. And the second actuator 64b is rotatable about a second stationary arm axis which is transverse to the first stationary arm axis. The first and second actuators 64a and 64b along with the lift 67 allow for full three-dimensional orientation of the robotic arm 40.

Returning to FIG. 2, the actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include respective actuators 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of the robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

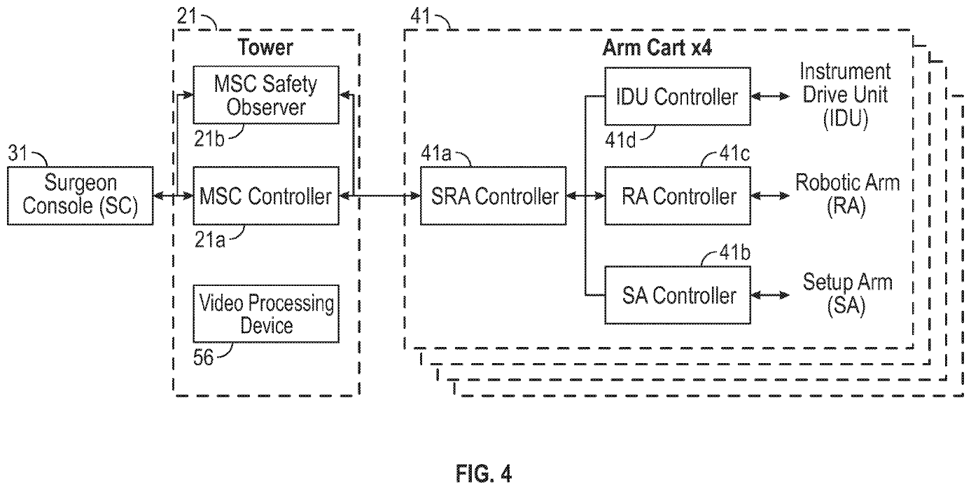

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

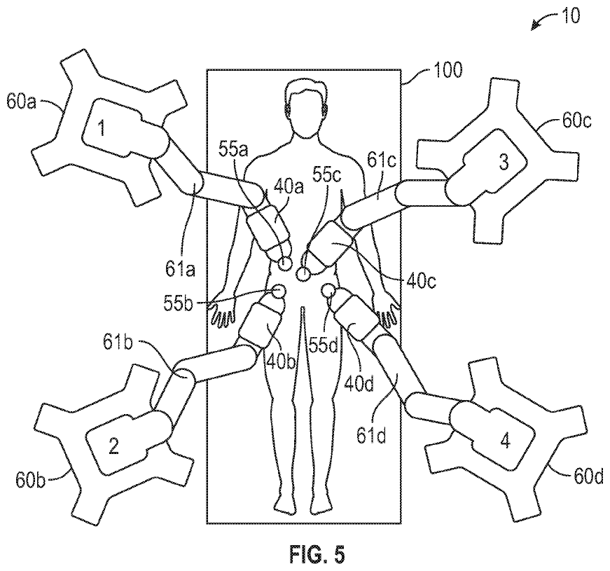

With reference to FIG. 5, the surgical robotic system 10 is setup around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

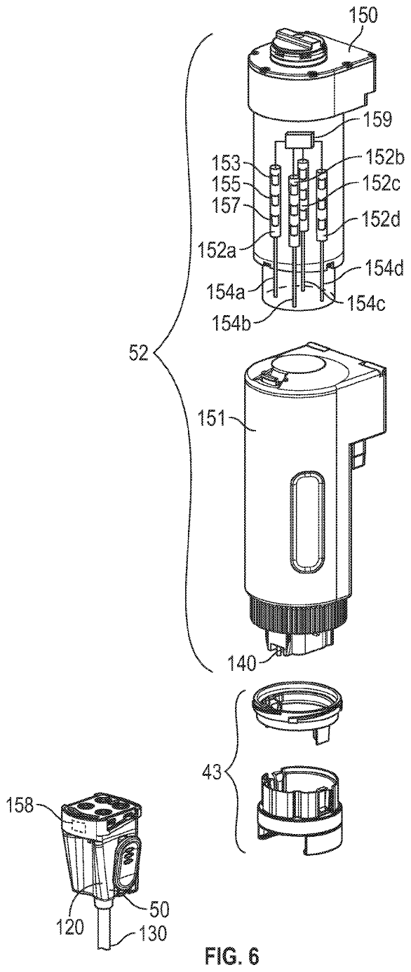

With reference to FIG. 6, the IDU 52 is shown in more detail and is configured to transfer power and actuation forces from its motors 152a-d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

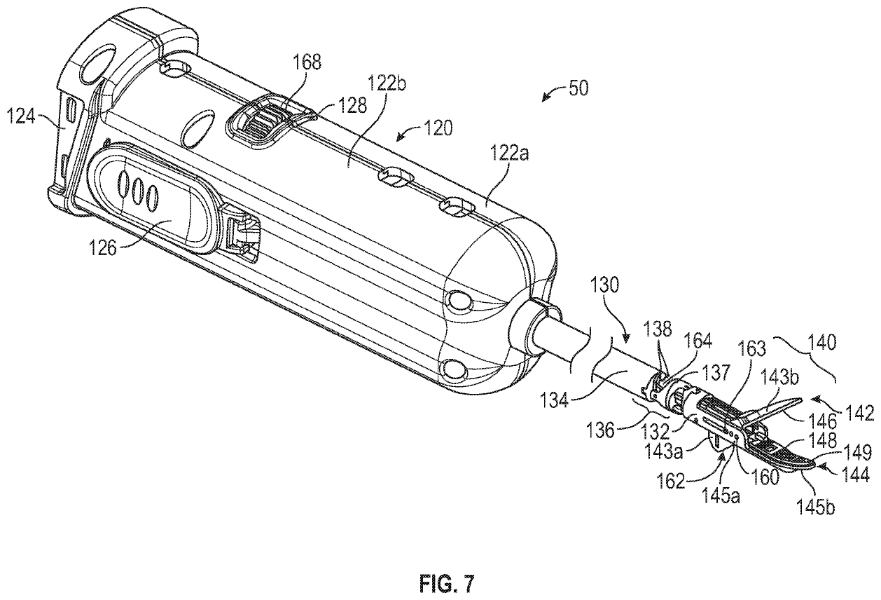

The IDU 52 includes a motor pack 150 and a sterile barrier housing 130. Motor pack 150 includes motors 152a-d for controlling various operations of the instrument 50. The instrument 50 is removably couplable to IDU 52. As the motors 152a-d of the motor pack 150 are actuated, rotation of the drive transfer shafts 154a, 154b, 154c, 154d of the motors 152a-d, respectively, is transferred to drive assemblies of the instrument 50. The instrument 50 is configured to transfer rotational forces/movement supplied by the IDU 52 (e.g., via the motors 152a-d of the motor pack 150) into longitudinal movement or translation of the cables or drive shafts to effect various functions of an end effector 200 (FIG. 7).

Each of the motors 152a-d includes a current sensor 153, a torque sensor 155, and a position sensor 157, which may be an angular motor position sensor. For conciseness only, operation of the motor 152a is described below. The sensors 153, 155, 157 monitor performance of the motor 152a. The current sensor 153 is configured to measure current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by motor 152a. Position sensor 157 may be any device that provides a sensor signal indicative of the number of rotations of the motor 152a, such as a mechanical encoder or an optical encoder. Parameters which are measured and/or determined by position sensor 157 may include speed, distance, revolutions per minute, position, and the like. Sensor signals from sensors 153, 155, 157 are transmitted to the IDU controller 41d (FIG. 4), which then controls the motors 152a-d based on the sensor signals, via an actuator controller 159. In particular, the actuator controller 159 controls torque outputted and angular velocity of the motors 152a-d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

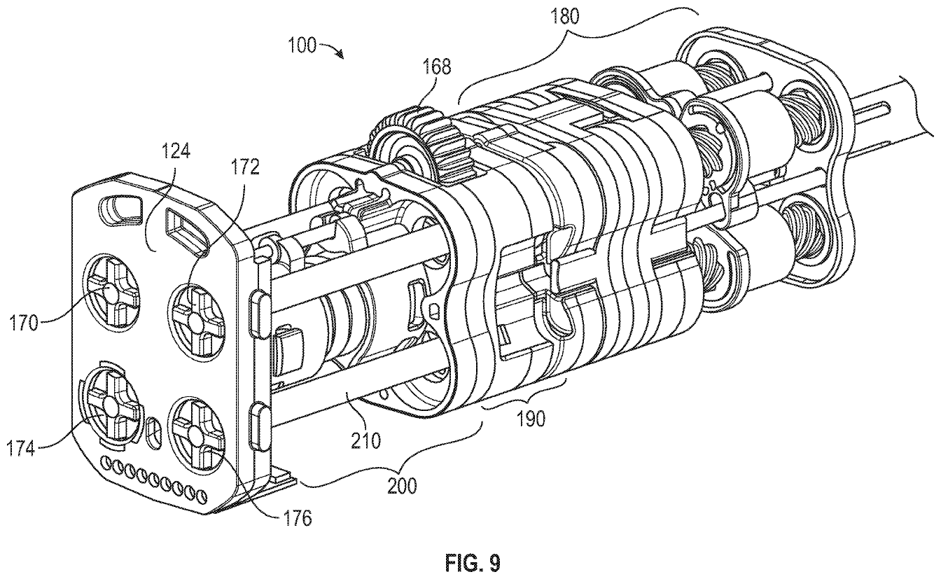

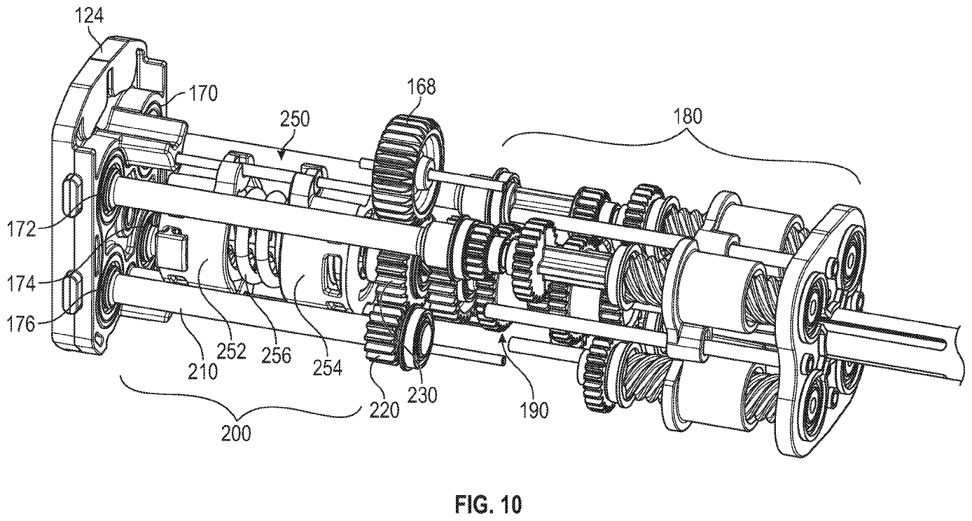

Referring to FIGS. 6-10, the instrument 50 includes the housing 120, a shaft 130 extending distally from housing 120, and end effector assembly 140 extending distally from shaft 130. A gearbox assembly 100 disposed within housing 120 and operably associated with end effector assembly 140. Housing 120 of instrument 50 is configured to selectively couple to IDU 52 of robotic, to enable motors 152a, 152b, 152c, 152d of IDU 52 to operate the end effector assembly 140 of the instrument 50. Housing 120 of instrument 50 supports a drive assembly that is mechanically actuated by the motors 152a, 152b, 152c, 152d of the IDU 52. Drive assembly of instrument 50 may include any suitable electrical and/or mechanical component to effectuate driving force/movement.

Instrument 50 is described herein as an articulating electrosurgical forceps configured for use with the robotic surgical system 10. However, the aspects and features of instrument 50 provided in accordance with the present disclosure, detailed below, are equally applicable for use with other suitable surgical instruments and/or in other suitable surgical systems.

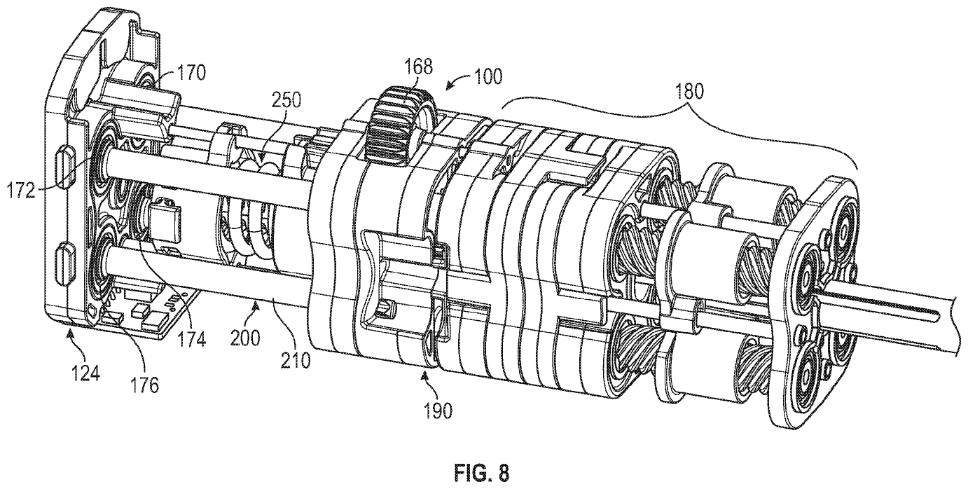

The housing 120 of instrument 50 includes first and second body portion 122a, 122b and a proximal face plate 124 that cooperate to enclose gearbox assembly 100 therein. Proximal face plate 124 includes apertures defined with couplers 170, 172, 174, 176 of gearbox assembly 100 extending through proximal face plate 124 (FIG. 8). A pair of latch levers 126 (only one of which is illustrated in FIG. 7) extend outwardly from opposing sides of housing 120 and enable releasable engagement of housing 120 with the IDU 52 of the robotic arm 40. An aperture 128 defined through housing 120 permits thumbwheel 168 to extend therethrough to enable manual manipulation of thumbwheel 168 from the exterior of housing 120 to permit manual opening and closing of end effector assembly 140.

Shaft 130 of instrument 50 includes a distal segment 132, a proximal segment 134, and an articulating section 136 disposed between the distal and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal segment 132 of shaft 130 at the distal ends thereof and extend proximally from distal segment 132 of shaft 130, through articulating section 136 of shaft 130 and proximal segment 134 of shaft 130, and into housing 120, wherein articulation cables 138 operably couple with an articulation sub-assembly 180 of gearbox assembly 100 to enable selective articulation of distal segment 132 (and, thus end effector assembly 140) relative to proximal segment 134 and housing 120, e.g., about at least two axes of articulation (e.g., yaw and pitch articulation). Articulation cables 138 may be arranged in a generally rectangular configuration, although other suitable configurations are also contemplated.

Articulation of end effector assembly 140 relative to proximal segment 134 of shaft 130, is accomplished by actuation of pair of cables 138. More specifically, in order to pitch end effector assembly 140, the upper pair of cables 138 are actuated while the lower pair of cables 138 are actuated relative to one another but in opposite manner relative to the upper pair of cables 138. With respect to yaw articulation, the right pair of cables 138 are actuated while the left pair of cables 138 are actuated but in opposite manner relative to the right pair of cables 138.

Continuing with reference to FIG. 7, end effector assembly 140 includes first and second jaw members 142, 144, respectively. Each jaw member 142, 144 includes a proximal flange portion 143a, 145a and a distal body portion 143b, 145b, respectively. Distal body portions 143b, 145b define opposed tissue-contacting surfaces 146, 148, respectively. Proximal flange portions 143a, 145a are pivotably coupled to one another about a pivot 160 and are operably coupled to one another via a cam-slot assembly 162 including a cam pin 163 slidably received within cam slots defined within the proximal flange portion 143a, 145a of at least one of the jaw members 142, 144, respectively, to enable pivoting of jaw member 142 relative to jaw member 144 and distal segment 132 of shaft 130 between a spaced-apart position (e.g., an open position of end effector assembly 140) and an approximated position (e.g. a closed position of end effector assembly 140) for grasping tissue between tissue-contacting surfaces 146, 148. As an alternative to this unilateral configuration, a bilateral configuration may be provided whereby both jaw members 142, 144 are pivotable relative to one another and distal segment 132 of shaft 130.

In embodiments, longitudinally extending knife channels 149 (only knife channel 149 of jaw member 144 is illustrated; the knife channel of jaw member 142 is similarly configured) are defined through tissue-contacting surfaces 146, 148, respectively, of jaw members 142, 144. In such embodiments, a knife assembly including a knife tube (not shown) extending from housing 120 through shaft 130 to end effector assembly 140 and a knife blade (not shown) disposed within end effector assembly 140 between jaw members 142, 144 is provided to enable cutting of tissue grasped between tissue-contacting surfaces 146, 148 of jaw members 142, 144, respectively. Knife tube (not shown) is operably coupled to a knife drive sub-assembly 190 of gearbox assembly 100 at a proximal end thereof to enable selective actuation thereof to, in turn, reciprocate the knife blade (not shown) between jaw members 142, 144 to cut tissue grasped between tissue-contacting surfaces 146, 148.

Referring to FIG. 7, a drive rod 164 is operably coupled to cam-slot assembly 162 of end effector assembly 140, e.g., engaged with the cam pin 163 thereof, such that longitudinal actuation of drive rod 164 pivots jaw member 142 relative to jaw member 144 between the spaced-apart and approximated positions. More specifically, urging drive rod 164 proximally pivots jaw member 142 relative to jaw member 144 towards the approximated or closed position while urging drive rod 164 distally pivots jaw member 142 relative to jaw member 144 towards the spaced-apart, open position. However, other suitable mechanisms and/or configurations for pivoting jaw member 142 relative to jaw member 144 between the spaced-apart and approximated positions in response to selective actuation of drive rod 164 are also contemplated. Drive rod 164 extends proximally from end effector assembly 140 through shaft 130 and into housing 120 wherein drive rod 164 is operably coupled with a jaw drive sub-assembly 200 of gearbox assembly 100 to enable selective actuation of end effector assembly 140 to grasp tissue therebetween and apply a closure force within an appropriate force range.

Tissue-contacting surfaces 146, 148 of jaw members 142, 144, respectively, are at least partially formed from an electrically conductive material and are energizable to different potentials to enable the conduction of electrical energy through tissue grasped therebetween, although tissue-contacting surfaces 146, 148 may alternatively be configured to supply any suitable energy, e.g., thermal, microwave, light, ultrasonic, ultrasound, etc., through tissue grasped therebetween for energy-based tissue treatment. Instrument 50 defines a conductive pathway (not shown) through housing 120 and shaft 130 to end effector assembly 140 that may include lead wires, contacts, and/or electrically-conductive components to enable electrical connection of tissue-contacting surfaces 146, 148 of jaw members 142, 144, respectively, to an energy source (not shown), e.g., an electrosurgical generator, for supplying energy to tissue-contacting surfaces 146, 148 to treat, e.g., seal, tissue grasped between tissue-contacting surfaces 146, 148.

With reference to FIGS. 8-10, the gearbox assembly 100 is disposed within housing 120 and includes an articulation sub-assembly 180, a knife drive sub-assembly 190, and a jaw drive sub-assembly 200. Articulation sub-assembly 180 is operably coupled between first and second couplers 170, 172, respectively, of gearbox assembly 100 and articulation cables 138 (FIG. 7) such that, upon receipt of appropriate inputs into first and/or second couplers 170, 172, articulation sub-assembly 180 manipulates cables 138 (FIG. 7) to articulate end effector assembly 140 in a desired direction, e.g., to pitch and/or yaw end effector assembly 140.

Knife drive sub-assembly 190 is operably coupled between third coupler 174 of gearbox assembly 100 and knife tube (not shown) such that, upon receipt of appropriate input into third coupler 174, knife drive sub-assembly 190 manipulates knife tube to reciprocate the knife blade (not shown) between jaw members 142, 144 to cut tissue grasped between tissue-contacting surfaces 146, 148.

Jaw drive sub-assembly 200, as detailed below, is operably coupled between fourth coupler 176 of gearbox assembly 100 and drive rod 164 such that, upon receipt of appropriate input into fourth coupler 176, jaw drive sub-assembly 200 pivots jaw members 142, 144 between the spaced-apart and approximated positions to grasp tissue therebetween and apply a closure force within an appropriate closure force range.

Gearbox assembly 100 is configured to operably interface with the IDU 52 when instrument 50 is mounted on robotic surgical system 10. That is, the motors 152a, 152b, 152c, 152d of IDU 52 selectively actuate couplers 170-176 of gearbox assembly 100 to articulate end effector assembly 140, grasp tissue between jaw members 142, 144, and/or cut tissue grasped between jaw members 142, 144. However, it is also contemplated that gearbox assembly 100 be configured to interface with any other suitable surgical system, e.g., a manual surgical handle, a powered surgical handle, etc.

With reference to FIGS. 8-10, jaw drive sub-assembly 200 of gearbox assembly 100 is shown generally including an input shaft 210, an input gear 220, a drive gear 230, the thumbwheel 168, a spring force assembly 250, and the drive rod assembly 164. The spring force assembly 250 includes a proximal hub 252, a distal hub 254, and a compression spring 256.

Compression spring 256 is disposed between proximal and distal hubs 252, 254 with a proximal portion thereof disposed within a cavity of proximal hub 252 and a distal portion thereof disposed within a cavity of distal hub 254. At least a portion of compression spring 256 is disposed about and/or configured to receive a portion of lead screw of drive gear 230 therethrough.

During use, jaw members 142, 144 are initially disposed in the spaced-apart position and, correspondingly, proximal and distal hubs 252, 254 are disposed in a distal-most position such drive rod 164 is disposed in a distal-most position. Further, in this position, compression spring 256 is disposed in a least-compressed condition; although even in the least-compressed condition, compression spring 256 may be partially compressed due to the retention of compression spring 256 in a pre-compressed configuration between proximal and distal hubs 252, 254.

In response to an input to close end effector assembly 140, e.g., rotational input to fourth (i.e., jaw) coupler 176 or a manual rotation of the thumbwheel 168, drive shaft 210 is rotated to thereby rotate input gear 220 which, in turn, rotates drive gear 230 such that distal hub 254 is translated proximally towards proximal hub 252. Proximal translation of distal hub 254 urges distal hub 254 against compression spring 256. Initially, where forces resisting approximation of jaw members 142, 144 are below a threshold corresponding to the spring value of compression spring 256, the closure force applied by jaw members 142, 144 is relatively low such that the urging of distal hub 254 proximally against compression spring 256 urges compression spring 256 proximally which, in turn, moves drive rod 164 proximally to pivot jaw member 142 relative to jaw member 144 from the spaced-apart position towards the approximated position to grasp tissue therebetween.

Upon further approximation of jaw members 142, 144 to grasp tissue therebetween, the forces resisting approximation of jaw members 142, 144, e.g., tissue resisting compression, may reach the threshold and, thus the closure force applied by jaw members 142, 144 may reach a corresponding threshold. In order to maintain the closure force applied by jaw members 142, 144 within a closure pressure range such as, for example, from about 3 kg/cm² to about 16 kg/cm², application of further closure force by jaw members 142, 144 is inhibited beyond this point despite further rotational input to fourth coupler 176. Once the threshold has been reached, further rotational input to fourth coupler 176 rotates drive shaft 210, input gear 220, and drive gear 230 to translate distal hub 254 further proximally into compression spring 256. However, rather than compression spring 256 urging proximal hub 252 further proximally to continue approximation of jaw members 142, 144 and increase the closure force applied therebetween, compression spring 256 is compressed, enabling proximal hub 252 and, thus, drive rod 164 to remain in position, thus inhibiting application of additional closure force between jaw members 142, 144. Operation of the instrument 50 and its components, including the compression spring 256, is described in more detail in U.S. Patent Application Publication No. 2020/0237453, filed on Jan. 29, 2019, the entire contents of which are incorporated by reference herein.

The surgical instrument 50 also includes a storage device 158 (FIG. 6). The storage device 158 includes non-volatile storage medium (e.g., EEPROM) that is configured to store any data pertaining to the surgical instrument 50, including but not limited to, usage count, identification information, model number, serial number, calibration data, and the like. In embodiments, the data may be encrypted and is only decryptable by the IDU controller 41d. The data may also be used by the IDU controller 41d to authenticate the surgical instrument 50. The storage device 158 may be configured in read only or read/write modes, allowing the IDU controller 41d to read as well as write data onto the storage device 158.

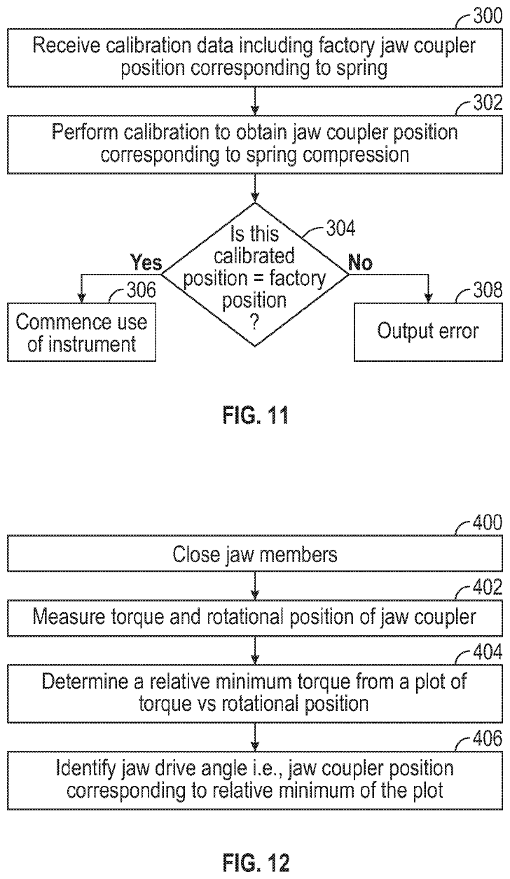

With reference to FIG. 11, a method for verifying operation of the instrument 50 may be embodied as software instructions executable by any one or more of the controllers of robotic system 10 (e.g., main controller 21a, the IDU controller 41d, etc.) The method includes receiving calibration data at the controller 21a at step 300. The calibration data may be stored in any suitable storage device locally (e.g., the storage device 158) or remotely, e.g., cloud, that is accessible by the robotic system 10. In embodiments where the calibration data is stored on the storage device 158, the IDU controller 41d may access the storage device 158 upon coupling of the instrument 50 to the IDU 52 either wirelessly or through an electrical connection between the instrument 50 and the IDU 52.

The method for performing the calibration process by the robotic system 10 prior to the use of the instrument 50 includes comparing the calibration data to stored calibration data that was generated during manufacture of the instrument 50. The process of generating calibration data during use of the instrument 50 or during manufacture is substantially the same and is described in further detail below with respect to FIG. 12.

Calibration data may be obtained during manufacture of the instrument 50, e.g., during of end of line calibration process, which may be performed using a calibration system configured to control and interface with the instrument 50 in a similar manner as the robotic arm 40 and the IDU 52. During calibration the instrument 50 is actuated across a range of motion for each degree of freedom of the instrument 50, e.g., pitch, yaw, rotation, jaw opening, etc., while various operational parameters, e.g., motor displacement, torque, current draw, etc., are measured and stored as calibration data.

During end of line calibration, the jaw members 142, 144 are closed on a load cell to a target jaw force, which may be from about 6.5 lbs. to about 7.0 lbs. During closure, the compression spring 256 in the instrument 50 is slightly compressed when the jaws are at a 0° angle i.e., fully closed. This ensures that there is spring compliance in the instrument 50 whenever a seal is made at any jaw angle.

Prior to determining the jaw closed position that achieves the desired jaw force, a check is done to find the position of the fourth jaw coupler 176 at which the compression spring 256 begins to compress at a 0° jaw angle/aperture, since the jaw members 142, 144 reach that position prior to the compression spring 256. This position is the minimum amount the instrument 50 is allowed to close while targeting the preset jaw force. Thus, calibration determines the compression point of the spring 256 based on a relationship between jaw force and jaw coupler 176 position (i.e., when the instrument 50 is in the spring compression region).

In order to find the minimum allowable jaw closed position, the instrument 50 is closed with nothing between the jaw members 142, 144 while measuring and recording torque imparted on fourth jaw coupler 176 as well as rotational displacement of the fourth jaw coupler 176. Rotational data may also be used to calculate linear displacement of the drive rod 164. The collected torque and position data may be smoothed using a running average method (e.g., 7×) and a second derivative (e.g., with a spread of 10). The position at the minimum value of the second derivative is also calculated and a safety factor, e.g., 2.5 degrees, may be added to this position to ensure that the spring is compressed at 0° jaw angle/aperture. This is the minimum jaw closed position value, which is then used by the robotic system 10 to operate the instrument 50. When the spring 256 is being compressed, the average relationship between jaw coupler positions and jaw force may be from about 5° to about 7° of jaw coupler rotations to increase jaw force by approximately 0.1 lbs.

FIG. 12 shows a method for calibrating the instrument 50, which may be embodied as software instructions executable by any one or more of the controllers of robotic system 10 (e.g., main controller 21a, the IDU controller 41d, etc.) or calibration platform described above.

At step 400, the instrument 50 is closed to a zero aperture (e.g., 0° jaw angle) while there is nothing between the jaw members 142, 144. At step 402, while the instrument 50 is closed and the spring 256 is compressed, torque and rotational displacement (i.e., position in degrees) of the fourth jaw coupler 176 is measured via the torque sensor 155 and the encoder sensor 157, respectively. The position data, i.e., the spring compression position and the jaw closed position, are stored in a memory of the calibration platform or the robotic system 10, depending on when calibration is being performed.

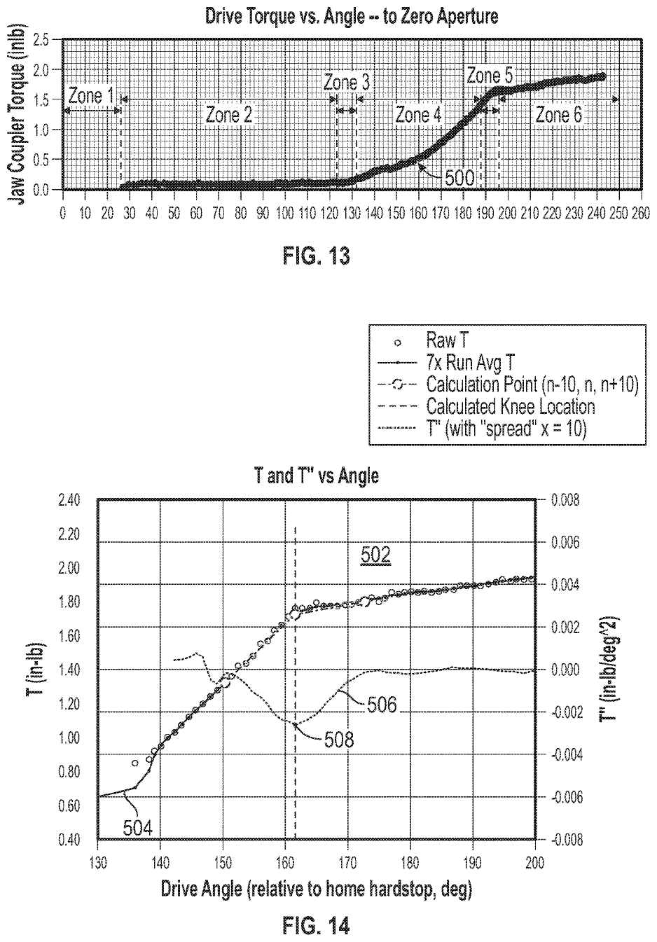

FIG. 13 shows a plot 500 of torque vs. position visualizing the data in six (6) zones. During zone 1 the fourth jaw coupler 176 is offset from hard stop (e.g., about) 0° to home position (e.g., about) 27°, at which motion of the jaw members 142, 144 is commenced. Zone 2 is a jaw closing zone, as the jaw members 142, 144 are approximated toward each other from an open configuration. The measured torque is very low until zone 3, which is a jaw contact zone, during which the jaw members 142, 144 contact each other and the torque begins to rise. Zone 4 is a system stiffness zone before the pre-load of the spring 256 is overcome. Zone 5 is a spring compression zone where the pre-load of the spring 256 is overcome and the spring 256 starts to compress. Zone 6 is a spring stiffness zone where the spring 256 is being compressed.

The position of the fourth jaw coupler 176 at zone 5 is used to set the minimum jaw closed position for each instrument 50 being calibrated as this value varies between different instruments. Zone 5 is a transition zone and is referred to as the “knee” in the curve due to its shape as shown in FIG. 14, which shows an enlarged portion 502 of the plot 500, showing the transition between zones 4 and 6.

The change in slope points of the plot 500 may be identified using minimum or maximum of the second derivative of the torque vs position plot 500. Thus, the change in slope point is a point at which there is a biggest change in slope of the plot 500. The largest change in slope is found when the second derivative is at a max or min (depending on the concavity of the curve). These second derivative minimums or maximums of the torque curve indicate a change of the state of the instrument 50 directly related to position of the jaw members 142, 144.

The position and torque data may be measured and recorded at a resolution of approximately one data point per degree of coupler rotation. In order to smooth out the data and eliminate transient noise (without excessively rounding off the knee location), a centered running average filter is applied to raw data. The filter may be a 7× or any other suitable filter as shown in Formula I, where Tis torque, and may be used to obtain filtered or smoothed data as illustrated by a running average plot 504 in FIG. 14.

$${T}_{n ( run avg )} = \frac{( {T}_{n - 3} + {T}_{n - 2} + {T}_{n - 1} + {T}_{n} + {T}_{n + 1} + {T}_{n + 2} + {T}_{n + 3} )}{7} \qquad ( I )$$

To find the transition in the running average plot 504, a second derivative of the plot 504 is determined at step 406. The location at which the torque vs drive angle slope changes (i.e., the transition of zone 5) corresponds to a relative minimum in the second derivative curve (i.e., going from a steep slope to a shallow slope). Formula II for a centered second derivative at point n, where torque (T) is a function of angular position (P).

$${T}_{n}^{″} = \frac{( \frac{( {T}_{n + 1} - {T}_{n} )}{( {P}_{n + 1} - {P}_{n} )} - \frac{( {T}_{n} - {T}_{n - 1} )}{( {P}_{n} - {P}_{n - 1} )} )}{( \frac{( {P}_{N + 1} - {P}_{n - 1} )}{2} )} \qquad ( II )$$

Formula (II) calculates the difference in slope between two-line segments (n−1 to n and n to n+1) divided by the distance between the center of these two “P” segments. However, even with a 7× running average to smooth out the data, there is still quite a bit of noise in the second derivative equation. Thus, at step 404, to further smooth out the second derivative equation and find the relative minimum, a wider spread between two data points may be used. Instead of calculating the second derivative at “n” using n−1 and n+1, a wider data set may be used, e.g., including data points n−10 and n+10, to generate a smoothed derivative plot 506. Using the wider spread, a relative minimum 508 at the transition point (i.e., spring compression point) is easily identifiable. The jaw drive angle position at the minimum 508 in the second derivative curve is the location at which the spring 256 begins to compress.

Zone 5 is the region in which the spring 256 begins to compress. The second derivative around zone 5 shows a minimum (i.e., changing from a steep slope to a shallow slope) at the change in slope point as shown in FIG. 14. This minimum is the knee location and indicates that the spring 256 in the instrument 50 is starting to compress.

In embodiments, an offset safety value, e.g., 2.5 degrees, may be added to this position as a factor of safety. This is the minimum jaw closed position value for the instrument 50 and is stored in the storage device 158 or any other suitable storage medium accessible by the robotic system 10.

With reference to FIG. 11, after receiving the calibration data, including the closed jaw position value for the instrument 50, the robotic system 10 performs a calibration process as described above with respect to the method of FIG. 12 at step 302 to obtain a second coupler position value. At step 304, the robotic system 10 compares the coupler position value received at step 300 to the newly obtained calibrated position value to determine whether they are substantially the same, i.e., if the difference between the two values is more than 0.1 turns.

If the values are the same, then the instrument 50 is operating in the same manner as at the time of its manufacture and the robotic system 10 proceeds at step 306 to using the instrument 50 during the procedure. If the values are not the same, the robotic system 10 at step 308 outputs an error, e.g., on one or more of the displays 23, 32, 34, and additionally may write a fault flag to the storage device 158 to prevent the use of the instrument 50 by any other robotic system 10. The verification method of FIG. 11 may be commenced automatically upon coupling of the instrument 50 to the IDU 52 or manually by the operator during the procedure, e.g., to verify the instrument 50 is functioning normally in response to a perceived state of failure.

With reference to FIG. 13, Zone 3 is the area at which the jaw members 142, 144 contact something (i.e., gross movement is stopped). Thus, a change in slope point at zone 3 may be used to identify the point at which the jaw members 142, 144 have contacted an obstruction, e.g., tissue. In this case, tissue contact may be determined by looking for a relative maximum (slopes going from shallow to steep) of the second derivative to find this change in slope point. Zone 3 may be used as an indicator of position of the jaw members 142, 144 and functionality of the instrument 50.

The system 10 calculates the second derivative of a torque vs position plot 500 and finds local minimum or maximum values to determine change in slope points (i.e., areas in which the behavior of the jaw members 142, 144 changes). These minimum or maximum values are directly related to positions at which the jaw members 142, 144 have stopped moving (i.e., zone 3) and positions at which the spring 256 of the instrument 50 starts to compress (i.e., zone 5). The zone 3 change in slope points may be used to confirm when the jaw members 142, 144 are gripping tissue and using this confirmation to provide real-time feedback to an electrosurgical generator or the operator regarding the size or stiffness of the vessel on which the the instrument 50 is clamped.

The changes in slopes of the torque vs position plot 500 may be used to confirm proper instrument behavior (i.e., during calibration). Furthermore, the change in slope points and deviations therefrom may be used to sense and verify instrument operation during a procedure using these same identification and comparisons of change in slope points of the torque vs position plot 500. It is envisioned that any mathematical method may be used to determine changes slope of the plot 500 besides using the second derivative.

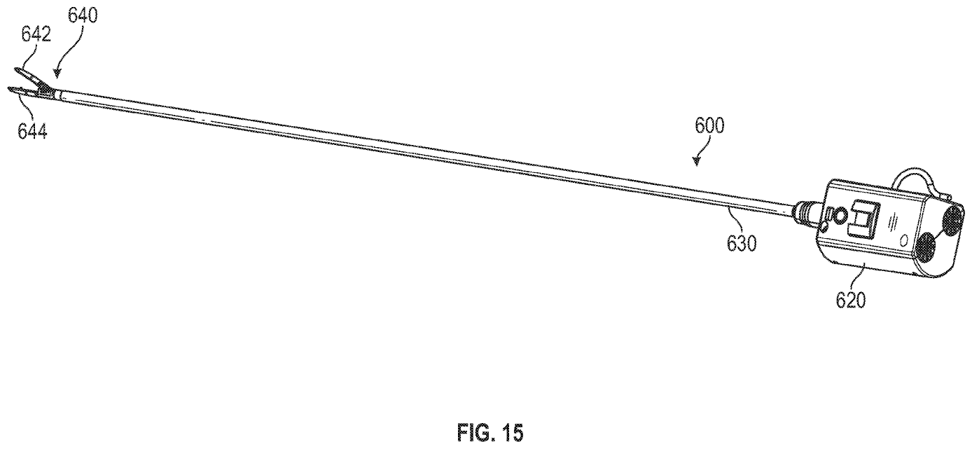

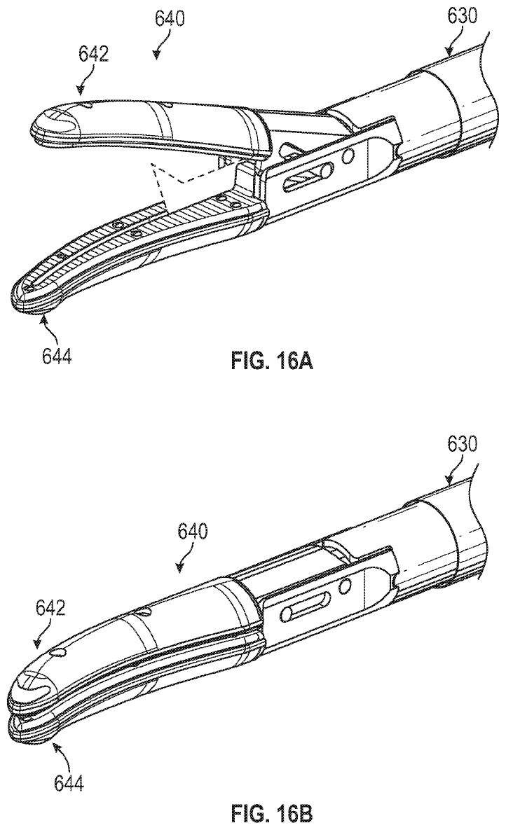

The disclosed system and method of calibration may be applied to any spring-loaded jawed instrument, such as an instrument 600 of FIG. 15, which is similar to the instrument 50 with some variations, such as lack of any articulation joints. This configuration minimizes the number of couplers that are being used and engaged by the IDU 52. With reference to FIGS. 15, 16A, and 16B, the instrument 600 includes a housing 620, a shaft 630 extending distally from housing 620, and end effector assembly 640 extending distally from shaft 630. The end effector assembly 640 also includes first and second jaw members 642 and 644, with the jaw member 642 being movable while the jaw member 644 is stationary relative to the shaft 630. The jaw member 642 may be actuated by a coupler 650 disposed at the distal end portion of the housing 620. For a more detailed description of the components of the instrument 600 and its operation reference may be made to U.S. Pat. No. 10,722,295, filed on Jan. 20, 2016, titled “Robotic surgical assemblies and electrosurgical instruments thereof,” the entire contents of which are incorporated by reference herein.

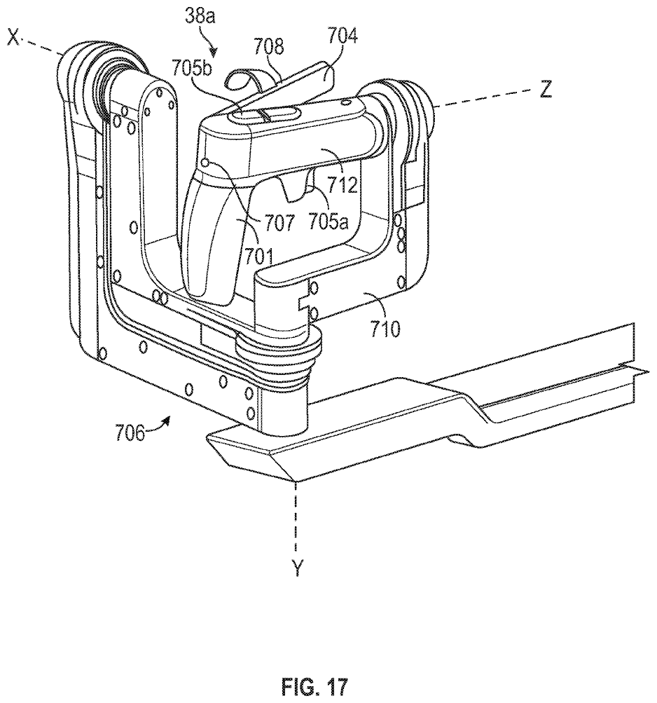

FIG. 17 shows the left-handle controller 38a, which is a mirror copy of the right-handle controller 38b. Each of the handle controllers 38a and 38b includes a handle 701 and a paddle 708 that is pivotally coupled to the handle 701 at one end (e.g., proximal) of the paddle 708. The paddle 708 is configured to control actuation, namely, opening and closing jaw members 142, 144 of the end effector assembly 140. The paddle 708 may include a finger sensor 704 configured to detect presence or movement of a finger, such as touch sensors, capacitive sensors, optical sensors, and the like. In embodiments, the finger sensor 704 may be disposed on any portion of the handle controllers 38a and 38b. Each of the handle controllers 38a and 38b may also include a trigger 705a and one or more buttons 705b for activating various functions of the instrument 50. In addition, each of the handle controllers 38a and 38b may include a gimbal assembly 706 allowing for movement and rotation of the handle controllers 38a and 38b about three axes (x, y, z). The handle controllers 38a and 38b may also include an infrared proximity sensor 707 configured to detect hand contact with a grip of the handle controllers 38a and 38b. The controller 31a of the surgeon console 30 monitors operator interactions with the handle controllers 38a and 38b and controls the instrument(s) 50 in response to operator inputs.

The paddle 708 is maintained, i.e., biased, in an open position by a feedback motor 712, which receives operator mechanical input, i.e., as the motor 712 is back driven during closure of the paddle 708 toward the closed position. The motor 712 also provides force feedback to the paddle 708 by counteracting operator's input, i.e., the motor 712 is forward driven. In addition, the motor 712 also measures the force, angle relative to the handle 701, and/or velocity of the paddle 708. The angle of the paddle 708 relative to the handle 701 is proportional to the angle between jaw members 142, 144. Thus, the paddle 708 and the jaw members 142, 144 may be fully aligned when in fully open and fully closed position and the jaw angle in between those position corresponds the paddle angle during the travel of the paddle 708.

In addition, the controller 31a also monitors individual or a new velocity of each joint of the gimbal assembly 706 as well as displacement of each of the joint of the gimbal assembly 706 and/or net displacement of the gimbal assembly 706. Details of the handle controllers 38a and 38b are provided in U.S. Patent Publication No. 2020/0315729, titled “Control arm assemblies for robotic surgical systems” filed on Nov. 30, 2018, the entire contents of which are incorporated by reference herein.

A feedback assembly 710 is disposed in the handle controller 38b to provide vibratory or haptic feedback to the operator. As shown, the feedback assembly 710 is configured to provide vibrational feedback at set frequencies and intervals to provide a sensation of touching. The feedback assembly 710 may include eccentric rotating mass (ERM) actuator, a linear resonant actuator (LRA), a piezoelectric actuator, or any other suitable tactile actuator configured to impart information to the operator through their sense of touch. Details of the haptic feedback mechanism are provided in U.S. Pat. No. 10,517,686, titled “Haptic feedback controls for a robotic surgical system interface” filed Apr. 13, 2018, the entire contents of which are incorporated by reference herein.

The paddle 708 is used to actuate various components of the instrument 50, e.g., open and close jaw members 142, 144. Thus, during use, the operator applies a constant force to close the jaw members 142, 144 from fully open to fully closed configuration. To maintain full jaw closure, the operator maintains force on the paddle 708 to ensure the jaw members 142, 144 are fully closed.

Robotic instruments 50 may have similar functionality as handheld surgical instruments, which may include a mechanical latch in the handle to maintain jaw closure. Such features allow the operator to retain jaw members in a closed position and provide the operator with tactile feedback. The present disclosure aims to maintain the same operator experience across robotic and handheld instruments by replicating the operator experience of handheld instruments on the robotic system 10. The is accomplished by simulating certain mechanical functionality via the hardware and software components of the robotic system 10.

The force provided by the motor 712 to the paddle 708 is ramped up as the jaw members 142, 144 move from the open position until a predetermined latch position is reached, which may correspond to the latch position of the counterpart handheld instrument. After the latch position is crossed, as the paddle 708 and jaw members 142, 144 are brought toward their respective closed positions. Force feedback provided to the paddle 708 is increased to maintain the paddle 708 in the latch position until the paddle 708 is fully closed prior to unlatching, thereby simulating the use of a mechanical instrument. When the operator latches the instrument 50, the jaw members 142, 144 remain at full closure until the surgeon unlatches by fully closing the paddle 708.

The present disclosure also provides force feedback through the paddle 708 based on the amount of force being applied to the tissue by the jaw members 142, 144. The controller 31a uses a force curve which expresses the feedback force as a function of the angle between the paddle 708 and the handle 701 (which also corresponds to the jaw angle) to determine the amount of force to be applied to the paddle 708. In particular, the controller 31a uses the open jaw position, determined during calibration process of the present disclosure, a real time determination of the “knee” location, determined via measured torque, and the close position, to determine the specific amount of force feedback.

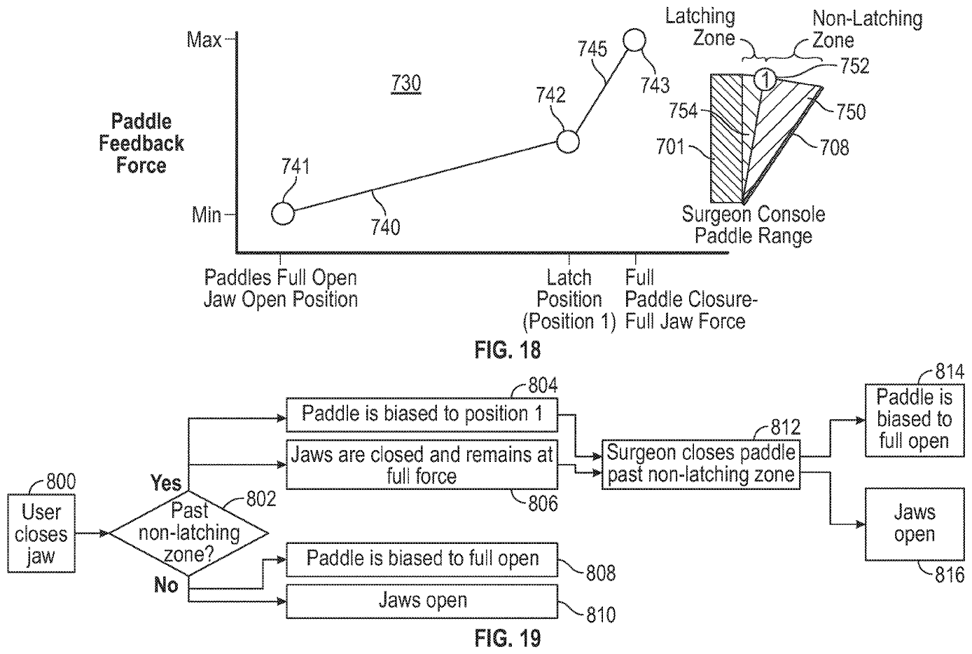

FIG. 18 shows a force feedback plot 730 that is implemented as software instructions executable by the controller 31a or any other controller of the system 10. FIG. 19 shows a method, which is also implemented as software instructions provided force feedback to the paddle 708 via the motor 712 to enable latching operation of the instrument 50. At step 800, the user commences closing jaw members 142, 144 by closing the paddle 708 toward the handle 701, which are schematically shown in FIG. 19. The paddle 708 is movable from a fully open position until a fully closed position. The movement range of the paddle 708 includes a non-latching zone 750 commencing from the fully open position and a latching zone 754 commencing from the fully closed position. In addition, a latch position 752 lies at a boundary between the non-latching zone 750 and a latching zone 754. The latch position 752 is used as a threshold for enabling latching operation.

During step 800, the force feedback is provided by the motor 712 to the paddle 708 according to a portion 740 of the plot 730. The portion 740 provides the force applied to the paddle 708 as a function of the travel of the paddle 708 through the non-latching zone 750. The portion 740 of the plot 730 extends from an opening point 741 to a latch point 742. The opening point 741 represents the jaw members 142, 144 and the paddle 708 being in a fully open position and a corresponding minimum force. The latch point 742 represents the latch position 752, during which a higher force is applied. As the paddle 708 and the jaw members 142, 144 travel between the opening and latch points 741, 742 the force applied to the paddle 708 may be increased in any suitable (e.g., linear, exponential, etc.) manner. The portion 740 may apply force in a linear manner and may have a first rate of change (e.g., slope).

At step 802, the controller 31a verifies whether the paddle 708 is past the latch position 752. If the paddle 708 is still in the non-latching zone 750, at step 804, the paddle 708 is biased into the fully open position by the motor 712 and at step 806 the jaw members 142, 144 are correspondingly opened by the IDU 52. If the paddle 708 is in the latching zone 754, then at step 808, the paddle 708 is biased to the latch position 752 by the motor 712 while the jaw members 142, 144 are closed at full force by the IDU 52 at step 810.

After the jaw members 142, 144 are fully closed, at step 812, the paddle 708 is moved from the latch position 752 to the fully closed position to the end of the latching zone 754. During step 812, the force feedback is provided by the motor 712 to the paddle 708 according to a portion 745 of the plot 730. The portion 745 provides the force applied to the paddle 708 as a function of the travel of the paddle 708 through the latching zone 754. The portion 745 of the plot 730 extends from the latch point 742 to a closure point 743. The closure point 743 represents the jaw members 142, 144 and the paddle 708 being in a fully closed position and a corresponding maximum force. As the paddle 708 and the jaw members 142, 144 travel between the second and closure points 742, 743 the force applied to the paddle 708 may be increased in any suitable (e.g., linear, exponential, etc.) manner. The portion 745 may apply force in a linear manner and may have a second rate of change (e.g., slope), that is larger than the rate of change of the first portion 740.

Once the paddle 708 is moved past the latch position 752 to fully closed position, the paddle 708 may be moved back to the latch position 752 and remain there until the paddle 708 is closed again to the fully closed position in order to unlatch the jaw members 142, 144. Latching maintains the jaw members 142, 144 in the closed position without having to maintain pressure on or closure of the paddle 708. The applied force to the paddle 708 is decreased according to the second portion 745 of the plot 730 while the paddle 708 is moved back from the fully closed position to the latch position 752. To unlatch the jaw members 142, 144, the paddle 708 is moved again from the latch position 752 to the fully closed position and the paddle 708 is then allowed to return, i.e., biased, to the open position. Unlatching occurs in steps 814 and 816, where the paddle 708 is biased into the fully open position and the jaw members 142, 144 are fully open.

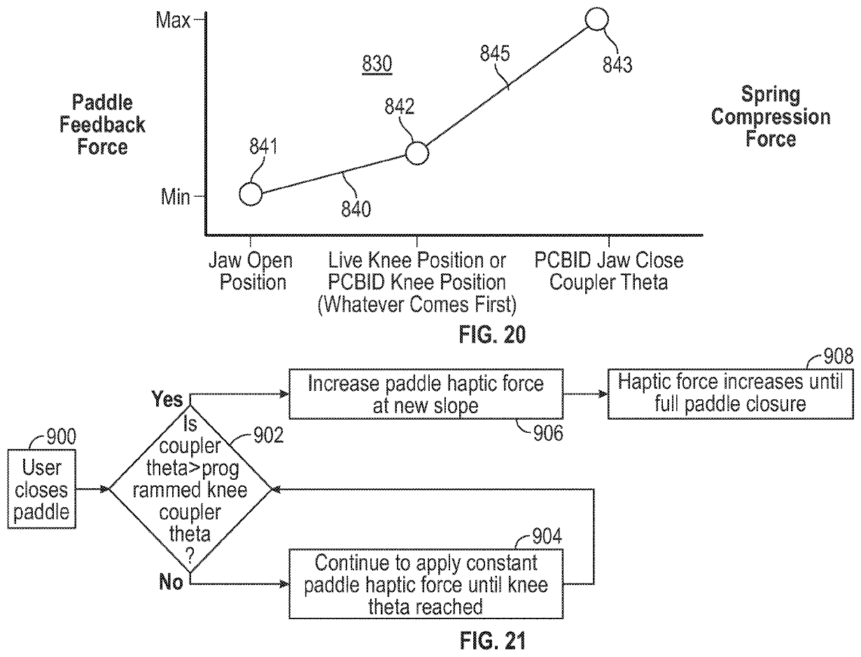

FIGS. 20 and 21 illustrate another method for applying force feedback to the paddle 708 based on rotational position of the fourth jaw coupler 176 that is responsible for closing the jaw members 142, 144. FIG. 20 shows a force feedback plot 830 that is implemented as software instructions executable by the controller 31a or any other controller of the system 10. FIG. 21 shows a method, which is also implemented as software instructions provided force feedback to the paddle 708 via the motor 712.

At step 900, the user commences closing jaw members 142, 144 by closing the paddle 708 toward the handle 701. The paddle 708 is movable from a fully open position until a fully closed position. During step 900, the force feedback is provided by the motor 712 to the paddle 708 according to a portion 840 of the plot 830. The portion 840 provides a value of the force that is applied by the motor 712 to the paddle 708 as a function of the travel of the paddle 708. The portion 840 of the plot 830 extends from an opening point 841 to a change in slope point 842. The opening point 841 represents the jaw members 142, 144 and the paddle 708 being in a fully open position and a corresponding minimum feedback force. The change in slope point 842 represents a point at which rotational position (e.g., 0) the fourth jaw coupler 176 has reached the calibrated coupler rotational position (i.e., “knee” location). As the paddle 708 and the jaw members 142, 144 travel between the opening and change in slope points 841, 842 the force applied to the paddle 708 may be increased in any suitable (e.g., linear, exponential, etc.) manner. The portion 840 may apply force in a linear manner and may have a first rate of change (e.g., slope), which may be constant (e.g., 0).

At step 902, the controller 31a compares whether the jaw coupler 176 has reached the calibrated coupler position (see FIGS. 12-14). If not, then at step 904, the controller 31a continues to apply the feedback force according to the portion 840 of the force feedback plot 830. Once the jaw coupler 176 position is past the calibrated position, then at step 906, the controller 31a increases the force until step 908 when the jaw members 142, 144 and the paddle 708 are fully closed.

Alternatively, rather than comparing whether the jaw coupler 176 has reached the calibrated coupler position, the controller 31a may analyze the torque during rotation of the jaw coupler 176 to identify a “knee” location, i.e., change in slope point in a torque vs rotational position plot (see step 406 of FIG. 12 and Zone 5, FIG. 13). Thus, at step 902, the controller 31a may monitor the torque vs rotational position to identify whether a change in slope point has occurred. If the inflection is not identified, then at step 904, the controller 31a continues to apply the feedback force according to the portion 840 of the force feedback plot 830. If the change in slope point has been identified, then at step 906, the controller 31a increases the haptic force until step 908 when the jaw members 142, 144 and the paddle 708 are fully closed.

During steps 906 and 908, the force feedback is provided by the motor 712 to the paddle 708 according to a portion 845 of the plot 830. The portion 845 provides a value of the force that is applied by the motor 712 to the paddle 708 as a function of the travel of the paddle 708 after the coupler has been rotated past the calibrated position or whether a change in slope point has occurred. During this movement of the paddle 708, the jaw members 142, 144 are closed while the spring 256 is being compressed. The portion 845 of the plot 830 extends from the change in slope point 842 to a closure point 843. The closure point 843 represents the jaw members 142, 144 and the paddle 708 being in a fully closed position and a corresponding maximum feedback force. As the paddle 708 and the jaw members 142, 144 travel between the inflection and closure points 842, 843 the force applied to the paddle 708 may be increased in any suitable (e.g., linear, exponential, etc.) manner. The portion 845 may apply force in a linear manner and may have a second rate of change (e.g., slope), that is larger than the rate of change of the first portion 840.

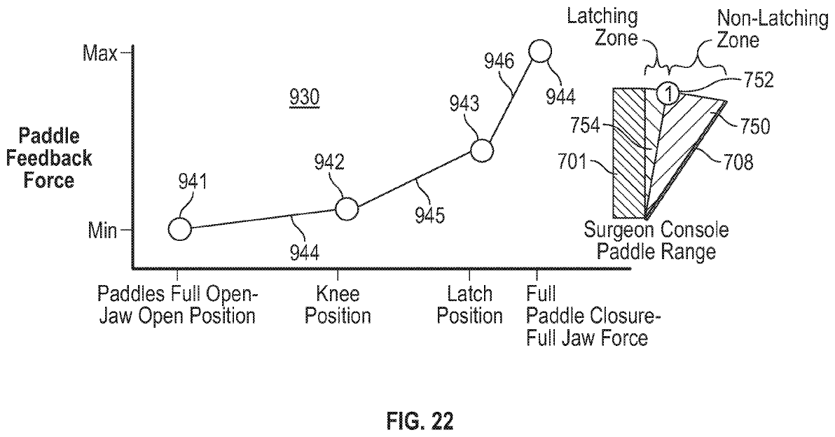

The methods of FIGS. 19 and 21 as their corresponding force feedback plots 730 and 830 of FIGS. 18 and 20 may be combined to provide for force feedback at different points of closure of the paddle 708. FIG. 22 shows a force feedback plot 930, which includes an opening point 941, a change in slope point 942, a latch point 943, and a closure point 944. The opening point 941 corresponds to the opening points 741 and 841 described above with respect to FIGS. 18 and 20. Thus, the opening point 841 represents the jaw members 142, 144 and the paddle 708 being in a fully open position and a corresponding minimum feedback force. The change in slope point 942 corresponds to the change in slope point 842, and represents a point at which rotational position (e.g., 0) the fourth jaw coupler 176 has reached the calibrated coupler position (i.e., “knee” location) or alternatively, the change in slope point as identified during the jaw closure process. The latch point 943 corresponds to the latch point 742 and represents the latch position 752. The closure point 944 corresponds to the final, and closure points 743 and 843 and represents the jaw members 142, 144 and the paddle 708 being in a fully closed position and a corresponding maximum feedback force. The points 941-944 are interconnected by portions 945, 946, 947 and provide a value of the force that is applied by the motor 712 to the paddle 708 as a function of the travel of the paddle 708 between the points 941-944. The portions 945-947 apply force in a linear manner and may have a progressively increasing rates of change (e.g., slopes). Transition between the points 941-944 may be done using the corresponding steps of the methods of FIGS. 19 and 21, i.e., determining whether latch point has been reached, whether change in slope point has been detected, etc.

Currently, jawed instruments, such as instruments 50 and 600, may be calibrated outside and inside the access port 55. External calibration is performed to determine the fully open and closed jaw position by moving their respective jaw members 142, 144 and 642, 644 to a fully open hard stop position, homing the knife, and moving the jaw members to the fully closed jaw position. During this process, the IDU controller 41d measures various parameters (e.g., torque, angular position, etc.) of the motors 152a-d using feedback from the sensors 153, 155, 157. The fully open and closed jaw positions may be determined by comparing the measured torque to a predetermined torque threshold while the motors 152a-d are moving the jaw members 142, 144 and 642, 644 to open and closed positions. Once the open hard stop is determined, fully closed jaw position is determined relative to the calibrated open hard stop.

Calibration may also be performed internally, i.e., inside a canula of the access port 55, to calibrate pitch, yaw, and articulation of the end effector assemblies 140 and 640. During this calibration, the end effector assembly is moved to contact the inside of the cannula to determine hard stops and zero position of pitch, yaw, and articulation degrees of freedom.

The present disclosure provides another method for calibrating open and close positions of spring-loaded jawed instruments. The novel calibration method is based on a position of the jaw members at which the spring compresses. As described above, a “knee” point, i.e., change in slope point in a torque vs rotational position plot (see step 406 of FIG. 12 and Zone 5, FIG. 13) may be used for calibration of such instruments.

When using open-to-closed calibration methods, end-of-line manufacturing systems are used to determine open and closed positions, which are then used during calibration of the instruments in the field by the robotic system 10. Thus, end-of-line test systems need to be closely aligned to the deployed robotic systems 10 that perform the calibration in the field. However, variability between different robotic systems 10, backlash differences, and homing variability, all play a role in how far the jaw members will be closed when used by different systems. Thus, there can be a lot of variability in determining the hard stop position and the fully closed jaw position when driving an instrument from an open hard stop to a predefined fully closed position.

The disclosed method reduces calibration variability and timing and improves alignment between end-of-line tests and use of the instruments by the robotic system 10. In particular, calibrating the instruments based on the “knee” location of the instrument coupler(s) (e.g., coupler 176) that controls compression of the spring 256 may eliminate system-to-system variation, such as backlash and may eliminate the need for external (i.e., end-of-line) calibration because there would be no need to find an open hard stop position.

Instead of driving jaw members from an open hard stop to a fully closed position, in the present calibration method, the jaws are controlled relative to the knee position, at which the spring 256 is compressed. An end-of-line test may be used to determine how far into the spring a device needs to close in order to achieve a desired jaw closure force. During use of the instrument, the robotic system 10 may identify the knee location of the spring-compressing coupler 176 and then close the jaws the same distance, by compressing the spring to achieve the desired jaw closure force. Since the coupler knee location is unique to each instrument, using this point provides a consistent calibration criterion and eliminates a variability inherent in prior calibration methods due to system-to-system variation including backlash variation and homing variability

The calibration method for the jaw positions according to the present disclosure includes performing external calibration to obtain fully open and closed hard stops. Closed jaw position denotes the spring 256 being compressed to impart desired closure force by the jaws. Initially, jaw member open hard stop position is identified by opening the jaw members until a torque threshold corresponding to the hard stop is detected. Additionally, the jaw member home position is identified, which is the position at which the jaws contact each other. This is done to confirm the jaw aperture is maximized. Further, the knife blade is also retracted to its home position. The fully closed position of the jaw members is calibrated by closing the jaw members and identifying the knee position at which the spring begins to compress. The closing continues until the desired closure pressure is reached. Following external jaw calibration, internal articulation calibration may be performed.

Internal calibration may include closing the jaw members to a torque below the spring compression force, i.e., spring 256 is not compressed while jaws are closed, and inserting the jaws into the cannula of the access port 55. Once inside, the internal articulation calibration is performed. Additionally, fully closed position calibration is also performed by compressing the spring further to identify the knee position and then to continue to close the jaws until the fully closed position is identified. Furthermore, the blade may be retracted to its home position to verify its functionality.

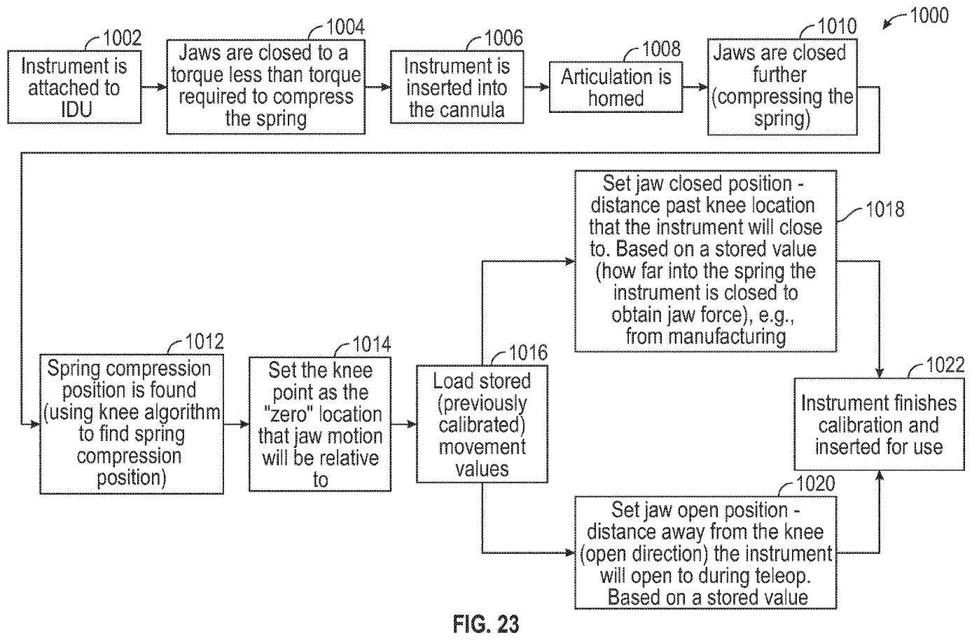

FIG. 23 shows a method 1000 for calibrating the instrument 50, which may be embodied as software instructions executable by any one or more of the controllers of robotic system 10 (e.g., main controller 21a, the IDU controller 41d, etc.) or calibration platform described above. The method of FIG. 23 may be used to calibrate any spring-loaded jawed instrument, such as instrument 600. At step 1002, the instrument 50 is coupled to the IDU 52 to initiate the calibration process.

At step 1004, the instrument 50 is closed to a zero aperture (e.g., 0° jaw angle) while there is nothing between the jaw members 142, 144. Jaw members 142 and 144 are closed to a torque that is less than the torque for compressing the spring 256. This is done so that the jaw members 142 and 144 are in contact with each other, while the spring 256 is not compressed. The motor torque to achieve this position of the jaw members 142, 144 may be about 20 Newton millimeters (Nmm).

At optional step 1006, the instrument 50, (including the end effector assembly 140) is inserted into the cannula of the access port 55 to continue calibration. As described above, the following calibration steps may be performed externally or internally of the cannula of the access port 55, thus, step 1006 may be optional.

At step 1008, the end effector assembly 140 is homed to 0° position for each of the articulating degrees of freedom, e.g., pitch and yaw. The articulation homing may be performed after articulation calibration to ensure the end effector assembly 140 is straight, namely, aligned along the same longitudinal axis as the shaft 130.

At step 1010, the jaw members 142, 144 are closed further, to compress the spring 256. During this process, at step 1012, the IDU controller 41d monitors torque and position of the motors 152a-d to determine the coupler knee location using a torque vs rotational position plot (see step 406 of FIG. 12 and Zone 5, FIG. 13). The coupler knee location is a change in slope point in the compression of the spring 256 and corresponds to position of the coupler 176 at which the spring 256 begins to compress while the jaw angle/aperture is at a 0°.

At step 1014, the IDU controller 41d sets the coupler position corresponding to the “knee” point as the calibrated zero position for movement of the jaw members 142, 144. Thus, any closure or opening movement is controlled relative to the calibrated zero position based on the “knee” point.

At step 1016, the IDU controller 41d loads one or more parameters for operating the instrument 50, which may be unique to the instrument 50. Parameters may be stored in any suitable device, such as flash memory disposed in the instrument 50, a cloud server, the memory of the robotic system 10, etc. Parameters include a closure distance value and an opening distance value. The distance values represent distances from the calibrated zero position to reach open and closed positions for the jaw members 142, 144. The distances may be determined during end-of-line testing or during any other previous calibration.

At step 1018, the IDU controller 41d sets the jaw closed position setpoint, which is calculated by adding the loaded closure distance value to the calibrated zero position (i.e., in the closing direction for the coupler). The jaw closed position setpoint is then used during operation of the instrument 50 as the maximal closure point to which the jaw members 142, 144 close.

At step 1020, the IDU controller 41d sets the jaw opened position setpoint, which is calculated by adding the loaded opening distance value to the calibrated zero position (i.e., in the opening direction for the coupler). The jaw opened position setpoint is then used during operation of the instrument 50 as the maximal opening point to which the jaw members 142, 144 open.

At step 1022, the jaw closure calibration is completed as the opening and closure setpoints are defined and used by the IDU controller 41d to control opening and closing of the jaw members 142, 144. After calibration, the instrument 50 is inserted through the access port 55 into the patient.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A method for calibrating a surgical robotic instrument, the method comprising:
actuating a motor to rotate a coupler of a surgical robotic instrument, wherein rotation of the coupler moves a drive rod disposed within the instrument in a longitudinal direction to compress a spring and approximate at least one jaw of a pair of opposing jaws of the instrument to a closed position;
measuring torque imparted by the motor and rotational position of the coupler during actuation of the motor to approximate the at least one jaw of the pair of opposing jaws of the instrument to the closed position;
determining a change in slope point from a plot of the torque and the rotational position;
identifying a coupler position corresponding to the change in slope point as a calibrated coupler position; and
controlling movement of the at least one jaw based on the calibrated coupler position.

2. The method according to claim 1, wherein the change in slope point is indicative of a start of compression of the spring.

3. The method according to claim 1, further comprising:
loading at least one calibration parameter for controlling movement of the at least one jaw based on the calibrated coupler position.

4. The method according to claim 3, further comprising setting a closed position setpoint based on the calibrated coupler position.

5. The method according to claim 4, wherein the at least one calibration parameter is a closure distance value.

6. The method according to claim 3, wherein setting the closed position setpoint includes adding the closure distance value to the calibrated coupler position.

7. The method according to claim 3, further comprising setting an opened position setpoint based on the calibrated coupler position.

8. The method according to claim 7, wherein the at least one calibration parameter is an opening distance value.

9. The method according to claim 8, wherein setting the opened position setpoint includes adding the opening distance value to the calibrated coupler position.

10. A surgical robotic system comprising:
a robotic arm including an instrument drive unit having a motor, a torque sensor, and a position sensor;
an instrument including:
a coupler configured to engage the motor;
a drive rod longitudinally movable by the coupler;
a spring compressed by the coupler during movement of the drive rod; and
a pair of opposing jaws movable by the drive rod to a closed position; and
a controller configured to:
actuate the motor to approximate the at least one jaw of the pair of opposing jaws to the closed position;
receive torque imparted by the motor from the torque sensor and rotational position of the coupler from the position sensor;
determine a change in slope point from a plot of the torque and the rotational position;
identify a coupler position corresponding to the change in slope point as a calibrated coupler position; and
control movement of the at least one jaw based on the calibrated coupler position.

11. The surgical robotic system according to claim 10, wherein the change in slope point is indicative of a start of compression of the spring.

12. The surgical robotic system according to claim 10, wherein the instrument further includes a storage device storing at least one calibration parameter.

13. The surgical robotic system according to claim 12, wherein the controller sets a closed position setpoint based on the calibrated coupler position.

14. The surgical robotic system according to claim 12, wherein the at least one calibration parameter is a closure distance value.

15. The surgical robotic system according to claim 14, wherein setting the closed position setpoint includes adding the closure distance value to the calibrated coupler position.

16. The surgical robotic system according to claim 12, wherein the controller sets an opened position setpoint based on the calibrated coupler position.

17. The surgical robotic system according to claim 16, wherein the at least one calibration parameter is an opening distance value.

18. The surgical robotic system according to claim 17, wherein setting the opened position setpoint includes adding the opening distance value to the calibrated coupler position.

19. A method for calibrating a surgical robotic instrument, the method comprising:
actuating a motor to rotate a coupler of a surgical robotic instrument, wherein rotation of the coupler moves a drive rod disposed within the instrument in a longitudinal direction to approximate at least one jaw of a pair of opposing jaws of the instrument until the pair of opposing jaws contact each other;
measuring torque imparted by the motor during actuation of the motor approximating the at least one jaw of the pair of opposing jaws of the instrument to the closed position;
determining the pair of opposing jaws contact each other based on measured torque;
actuating the motor to rotate the coupler of the surgical robotic instrument, wherein rotation of the coupler moves the drive rod to compress a spring and approximate the at least one jaw of the pair of opposing jaws of the instrument to a closed position;
measuring torque imparted by the motor and rotational position of the coupler during actuation of the motor approximating the at least one jaw of the pair of opposing jaws of the instrument to the closed position and compressing the spring;
determining a change in slope point from a plot of the torque and the rotational position;
identifying a coupler position corresponding to the change in slope point as a calibrated coupler position; and
controlling movement of the at least one jaw based on the calibrated coupler position.

20. The method according to claim 19, further comprising:
loading a closure distance value and an opening distance value;
setting a closed position setpoint by adding the closure value to the calibrated coupler position;
setting an opened position setpoint by adding the opening value to the calibrated coupler position; and
controlling movement of the pair of opposing jaw members between the closed position setpoint and the opened position setpoint.
